(19) United States
(12) Patent Application Publication
(10) Pub. No.: US 2024/0293493 A1
(43) Pub. Date: Sep. 5, 2024

(54) COMPOSITION FOR ALLEVIATING, TREATING, OR PREVENTING MUSCULAR DISEASES, OR IMPROVING MUSCULAR FUNCTIONS, CONTAINING KOREAN MINT EXTRACT OR TILIANIN AS ACTIVE INGREDIENT

(71) Applicant: AAT COSTECH CO., LTD., Seoul (KR)

(72) Inventors: Jae Kwan HWANG, Seoul (KR); Changhee KIM, Gyeonggi-do (KR); Yu Kyong WOO, Seoul (KR)

(73) Assignee: AAT COSTECH CO., LTD., Seoul (KR)

(21) Appl. No.: 18/002,740

(22) PCT Filed: Mar. 11, 2021
(86) PCT No.: PCT/KR2021/003053

§ 371 Date: Jun. 21, 2023

(30) Foreign Application Priority Data
Jun. 21, 2020 (KR) 10-2020-0075398
Mar. 11, 2021 (KR) 10-2021-0031725

(51) Int. Cl. A61K 36/532; A21D 2/36; A23G 3/48; A23G 4/06; A23L 2/52; A61K 8/9789; A61K 31/7048; A61P 21/00; A61Q 19/00

(52) U.S. Cl. CPC A61K 36/532; A21D 2/36; A23G 3/48; A23G 4/068; A23L 2/52; A61K 8/9789; A61K 31/7048; A61P 21/00; A61Q 19/00; A61K 2236/331; A61K 2236/333; A61K 2236/35; A61K 2236/37; A61K 2800/10

(57) ABSTRACT

The present disclosure relates to a composition for alleviating, treating or preventing muscular diseases or improving muscular functions, which contains a Korean mint extract or tilianin as an active ingredient. The Korean mint or tilianin according to the present disclosure has effects of increasing the activity of mTOR, which is involved in muscle protein synthesis, and reducing mRNA expression of MuRF-1 and atrogin-1, which are involved in muscle protein degradation. Accordingly, it can be usefully used for prevention, amelioration or treatment of various muscular diseases such as sarcopenia, muscular atrophy, muscular dystrophy, atony, muscular degeneration, myasthenia, cachexia, etc. by increasing muscle mass and improving muscular functions.

### TECHNICAL FIELD

The present disclosure relates to a composition for alleviating, treating or preventing muscular diseases or improving muscular functions, more particularly to a composition for alleviating, treating or preventing muscular diseases or improving muscular functions, which contains a Korean mint extract or tilianin as an active ingredient.

### BACKGROUND ART

Muscle atrophy refers to weakening and degeneration of muscles caused by gradual decrease of muscle mass (Cell, 119(7): 907-910, 2004). Muscle atrophy is facilitated by immobility, oxidative stress and chronic inflammation, and weakens muscular functions and motor ability (Clinical Nutrition, 26(5): 524-534, 2007). The most important factor that determines muscular functions is muscle mass, which is maintained by the balance of protein synthesis and degradation. Muscle atrophy occurs when protein degradation exceeds protein synthesis (The International Journal of Biochemistry and Cell Biology, 37(10): 1985-1996, 2005).

Muscle size is controlled by the intracellular signaling pathways that lead to anabolism or catabolismin muscles. When signaling for the synthesis of muscle proteins exceeds that for the degradation of muscle proteins, muscle protein synthesis is increased, resulting in increased muscle size (hypertrophy) or increased number of muscle fibers (hyperplasia) due to increased muscle proteins (The Korea Journal of Sports Science, 20(3): 1551-1561, 2011).

The factors involved in muscle protein synthesis trigger protein synthesis by phosphorylating proteins downstream of the phosphatidylinositol-3 kinase (PI3K)/Akt pathway in muscle cells. The activation of the mammalian target of rapamycin (mTOR) by PI3K/Akt signaling is recognized as a major growth signaling mechanism that integrates various intracellular growth signals. mTOR contributes to the increase of muscle mass by inducing muscle protein synthesis through activation of two factors that initiate mRNA translation, 4E-binding protein (4EBP1) and phosphorylated 70-kDa ribosomal S6 kinase (p70S6K) (The Korea Journal of Sports Science, 20(3): 1551-1561, 2011; The International Journal of Biochemistry and Cell Biology, 43(9): 1267-1276, 2011). Conversely, when forkhead box (FoxO), which is a transcription factor, migrates from the cytoplasm to the nucleus, the expression of the E3 ubiquitin ligase factors atrogin-1 and MuRF1 involved in protein degradation is increased (Disease Models and Mechanisms, 6: 25-39, 2013). If their expression level is increased, muscle mass is reduced as protein degradation in the muscle is increased. Thus, the activation of mTOR and inhibition of atrogin-1 and MuRF1 expression increases muscle mass by increasing the amount of muscle proteins.

Muscle cell differentiation and muscle formation are controlled by a variety of muscle regulatory factors. Among them, the expression of myogenin induced by the activation of MyoD is involved in the formation of myotubes, which is the most important factor in fusion of myoblasts. The muscle fibers formed through this process form bundles and finally forms muscles (Cellular and Molecular Life Sciences, 70: 4117-4130, 2013).

Korean mint (Agastache rugosa) is a plant belonging to the family Lamiaceae. It is reported to have antibacterial (Korean J. Medicinal Crop Sci., 10(3): 206-211, 2002), anti-inflammatory (J. Nat. Prod., 82, 12: 3379-3385, 2019), antioxidant (J. Korean Soc. Agric. Chem. Biotechnol., 42(3): 262-266, 1999), antiviral (Arch Pharm. Res., 22(5): 520-523, 1999), anti-obesity (J. Korean Med. Obes. Res., 15(2): 104-110, 2015) and antiaging (J. Microbiol. Biotechnol. 29(9): 1349-1360, 2019) activities.

Tilianin is a representative flavone compound found in Korean mint. It is reported to have antihypertensive (Biochem. Pharmacol., 78: 54-61, 2009), antioxidant (J Pharmacol Sci., 139: 352-360, 2019), anti-inflammatory (Front. Physiol., 10: 825, 2019), antidiabetic (Biomed. Phamacother., 83: 667-675, 2016), antiallergic (Phytomedicine, 80: 153392, 2021), anti-anxiety (Asian Pac J Trop Med, 8(3): 185-90, 2015), kidney-protecting (Int Immunopharmacol, 88: 106967, 2020) and anticancer (Front Pharmacol, 11: 205, 2020) activities.

However, nothing is known about the effect of ameliorating, treating or preventing muscular diseases or improving muscular functions of a Korean mint extract or tilianin yet.

### REFERENCES OF RELATED ART

### Patent Documents

Korean Patent Registration No. 1766373.

### DISCLOSURE

### Technical Problem

The inventors of the present disclosure have searched for a natural substance that has a superior activity or regulating muscular functions and can be used safely. As a result, they have identified that a Korean mint extract or tilianin has the activity of ameliorating, treating or preventing muscular diseases or improving muscular functions, and have completed the present disclosure.

The present disclosure is directed to providing a food composition for ameliorating or preventing muscular diseases or improving muscular functions, which contains a Korean mint extract or tilianin of Chemical Formula 1 as an active ingredient.

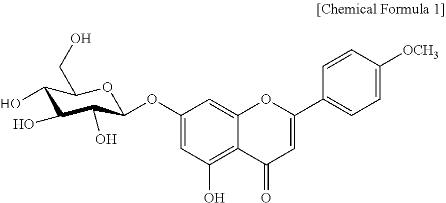

The present disclosure is also directed to providing a pharmaceutical composition for preventing or treating muscular diseases, which contains a Korean mint extract or tilianin of Chemical Formula 1 as an active ingredient.

The present disclosure is also directed to providing a cosmetic composition for ameliorating or preventing muscular diseases or improving muscular functions, which contains a Korean mint extract or tilianin of Chemical Formula 1 as an active ingredient.

The present disclosure is also directed to providing a feed additive for ameliorating or preventing muscular diseases or improving muscular functions, which contains a Korean mint extract or tilianin of Chemical Formula 1 as an active ingredient.

### Technical Solution

The present disclosure provides a food composition for ameliorating or preventing muscular diseases or improving muscular functions, which contains a Korean mint extract or tilianin of Chemical Formula 1 as an active ingredient.

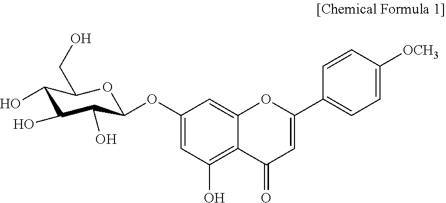

The present disclosure also provides a pharmaceutical composition for preventing or treating muscular diseases, which contains a Korean mint extract or tilianin of Chemical Formula 1 as an active ingredient.

The present disclosure also provides a cosmetic composition for ameliorating or preventing muscular diseases or improving muscular functions, which contains a Korean mint extract or tilianin of Chemical Formula 1 as an active ingredient.

The present disclosure also provides a feed composition for ameliorating or preventing muscular diseases or improving muscular functions, which contains a Korean mint extract or tilianin of Chemical Formula 1 as an active ingredient.

The present disclosure also provides a use of a pharmaceutical composition containing a Korean mint extract or tilianin of Chemical Formula 1 as an active ingredient for preparation of a pharmaceutical drug for preventing or treating muscular diseases.

The present disclosure also provides a method for treating a muscular disease, which includes administering a pharmaceutical composition containing a Korean mint extract or tilianin of Chemical Formula 1 as an active ingredient to a patient with a muscular disease.

### Advantageous Effects

A Korean mint extract or tilianin according to the present disclosure has a superior effect of increasing the activity of mTOR, which is involved in muscle protein synthesis, and inhibiting the mRNA expression of MuRF1 and atrogin-1, which are involved in muscle protein degradation. In addition, it can prevent, treat or ameliorate the decline of muscular functions, muscle loss, etc. by increasing muscle mass and improving muscular functions. Because the Korean mint extract or tilianin is a natural substance, it can be safely used for food, pharmaceutical drugs, cosmetics or feed additives without side effects.

### BRIEF DESCRIPTION OF DRAWINGS

FIG. 1 shows a result of investigating the increase in the activity of mTOR in L6 muscle cells by treatment with a hot water extract of an aerial part of Korean mint.

FIG. 2 shows a result of investigating the decrease in the mRNA expression level of MuRF1 and atrogin-1 in L6 muscle cells by treatment with a hot water extract of an aerial part of Korean mint.

FIG. 3 shows a result of investigating the increase in the activity of mTOR in L6 muscle cells by treatment with tilianin.

FIG. 4 shows a result of investigating the decrease in the mRNA expression level of MuRF1 and atrogin-1 in L6 muscle cells by treatment with tilianin.

FIG. 5 shows a result of investigating the increase of muscle volume in muscle atrophy-induced mice by treatment with a Korean mint extract.

### BEST MODE

Hereinafter, the present disclosure is described specifically.

The present disclosure provides a food composition for ameliorating or preventing muscular diseases or improving muscular functions, which contains a Korean mint extract or tilianin, a pharmaceutical composition for treating or preventing muscular diseases, which contains a Korean mint extract or tilianin, a cosmetic composition for ameliorating or preventing muscular diseases or improving muscular functions, which contains a Korean mint extract or tilianin, a feed additive for ameliorating or preventing muscular diseases or improving muscular functions, which contains a Korean mint extract or tilianin, or a method for treating a muscular disease, which includes applying a Korean mint extract or tilianin to human or a non-human mammal.

In the present specification, the ‘Korean mint’ refers to a perennial plant belonging to the family Lamiaceae. The whole plant of Korean mint, specifically an aerial part or leaf of Korean mint, may be used.

In the present specification, the ‘Korean mint extract’ refers to one obtained by extracting Korean mint using a solvent, and includes all of an extract, a diluted or concentrated extract, a fraction of the extract, a dried product obtained by drying the extract, or a crude purified product or a purified product thereof. The Korean mint extract may be obtained by extracting Korean mint with one or more solvent selected from a group consisting of water, a C₁-6 organic solvent and an ultra-high-pressure subcritical or supercritical fluid, although not being limited thereto.

The Korean mint extract of the present disclosure may be prepared by an extraction method commonly used in the art, such as hot extraction, cold extraction, ultrasonic extraction, filtration, reflux extraction, etc., and the Korean mint may be one which has been purchased commercially, harvested from the nature, or cultivated.

The Korean mint extract according to the present disclosure may be separated from the solvent according to a method commonly used in the art for preparation of an extract from a natural product, e.g., under common temperature and pressure conditions.

The term “fraction” used in the present specification refers to a product obtained by fractionating a mixture containing various ingredients into specific ingredients or groups thereof. In the present disclosure, it refers to a product obtained by fractionating a Korean mint extract into specific ingredients or groups thereof.

In order to obtain the Korean mint fraction according to the present disclosure, a fractionation solvent known in the art, e.g., a polar solvent such as an anhydrous or hydrous C₁-4 lower alcohol like water, ethanol, methanol, etc., a nonpolar solvent such as hexane, butanol, ethyl acetate, chloroform, dichloromethane, etc. or a mixture solvent thereof, may be used, although not being limited thereto.

The Korean mint fraction of the present disclosure may also include one that has passed through a purification process. For example, a fraction obtained by passing the Korean mint extract according to the present disclosure through an ultrafiltration membrane having a specific molecular weight cut-off value, or a fraction obtained by various purification methods such as various chromatographic techniques (for separation based on size, charge, hydrophobicity or affinity) is included in the Korean mint extract of the present disclosure.

In the present specification, the ‘tilianin’ includes a compound represented by Chemical Formula 1 or pharmaceutically acceptable salt thereof.

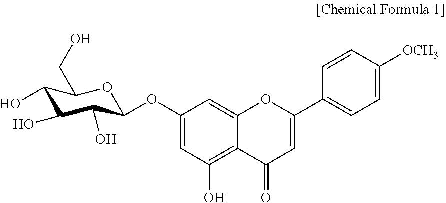

The tilianin of the present disclosure is a representative physiologically active ingredient existing in Korean mint. It can be obtained by isolation and purification from Korean mint, or can be synthesized or purchased.

In the present specification, the ‘muscle’ collectively refers to tendon, muscle and ligament. The ‘muscular function’ refers to the ability of exerting power through muscle contraction, and includes muscular strength which refers to the ability of exerting maximum muscle power to resist resistance, muscular endurance which refers to the ability of the muscle to sustain repeated contractions against a given weight for an extended period of time, and explosive muscular strength which refers to the ability of exerting strong power in short time. In the present specification, the term ‘improving muscular functions’ refers to improving muscular functions by increasing muscle mass.

The present disclosure provides a food composition for ameliorating or preventing muscular diseases or improving muscular functions, a pharmaceutical composition for preventing or treating muscular diseases, a cosmetic composition for ameliorating or preventing muscular diseases or improving muscular functions, or a feed additive for ameliorating or preventing muscular diseases or improving muscular functions, which contains a Korean mint extract or tilianin as an active ingredient.

In a specific exemplary embodiment, the Korean mint extract may be a hot water extract, an ethanol extract, an ethyl acetate extract, a hexane extract, an ultra-high-pressure extract, a subcritical extract or a supercritical extract, although not being limited thereto.

In a specific exemplary embodiment, the Korean mint extract may be obtained by extracting the aerial part or leaf of Korean mint with one or more solvent selected from a group consisting of water, a C₁₋₆ organic solvent and a subcritical or supercritical fluid. In addition, it may also be obtained by extracting Korean mint under an ultra-high-pressure condition of 100 MPa or higher. If necessary, it may be prepared by including an additional step of filtration or concentration according to a method known in the art.

In a specific exemplary embodiment, the C₁₋₆ organic solvent may be one or more selected from a group consisting of a C₁₋₆ alcohol, acetone, ether, benzene, chloroform, ethyl acetate, methylene chloride, hexane, cyclohexane and petroleum ether.

In a specific example of the present disclosure, the inventors of the present disclosure have prepared an extract of the aerial part or leaf of Korean mint by extracting a dried and pulverized aerial part or leaf of Korean mint by repeated extraction at room temperature using ethanol, ethyl acetate or hexane as a solvent, hot water extraction, ultra-high-pressure extraction, subcritical fluid extraction or supercritical fluid extraction.

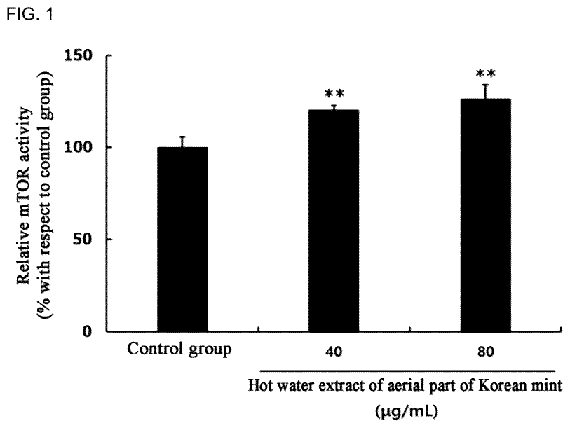

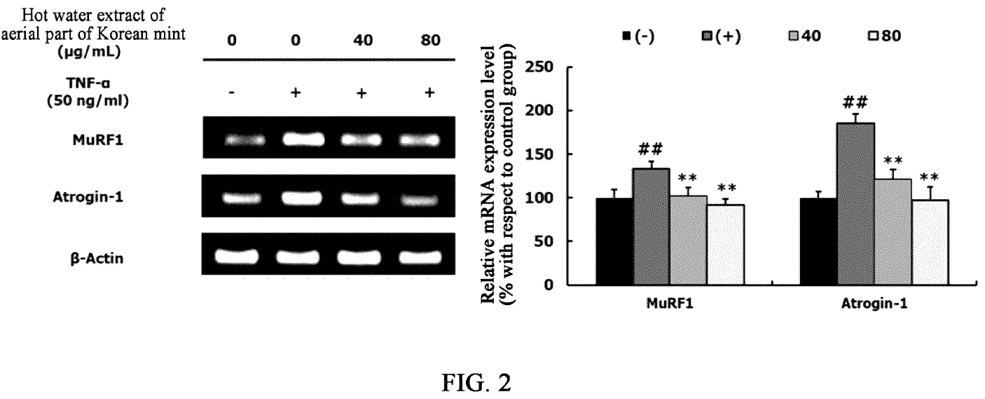

As a result of treating L6 muscle cells with the extract of the aerial part or leaf of Korean mint, it was confirmed that the expression of the p-mTOR protein involved in muscle protein synthesis was increased significantly (FIG. 1, Table 1 and Table 3). In addition, when L6 muscle cells were treated with the extract of the aerial part or leaf of Korean mint, it was confirmed that the mRNA expression of MuRF-1 and atrogin-1, which are involved in muscle protein degradation, was inhibited significantly (FIG. 2, Table 2 and Table 4).

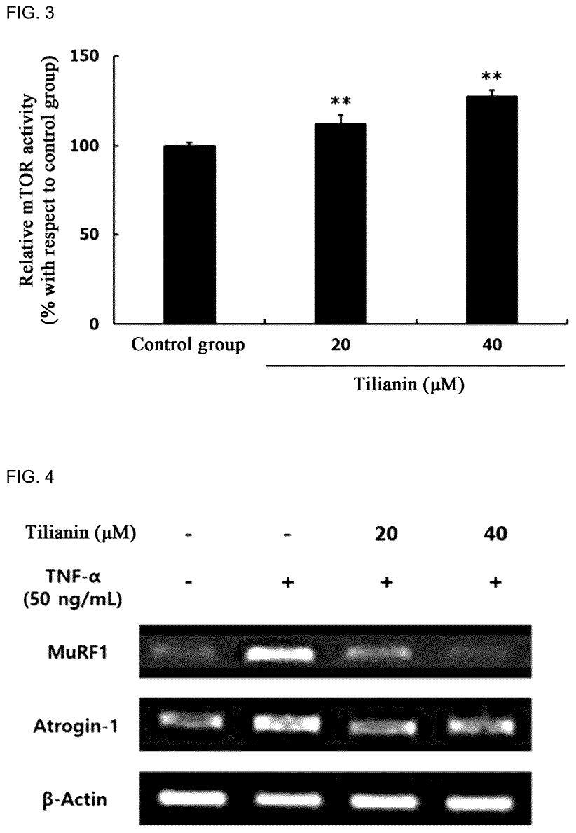

When L6 muscle cells were treated with tilianin, it was confirmed that the expression of the p-mTOR protein involved in muscle protein synthesis was increased significantly (FIG. 3). In addition, when L6 muscle cells were treated with tilianin, it was confirmed that the mRNA expression of MuRF-1 and atrogin-1, which are involved in muscle protein degradation, was inhibited significantly (FIG. 4).

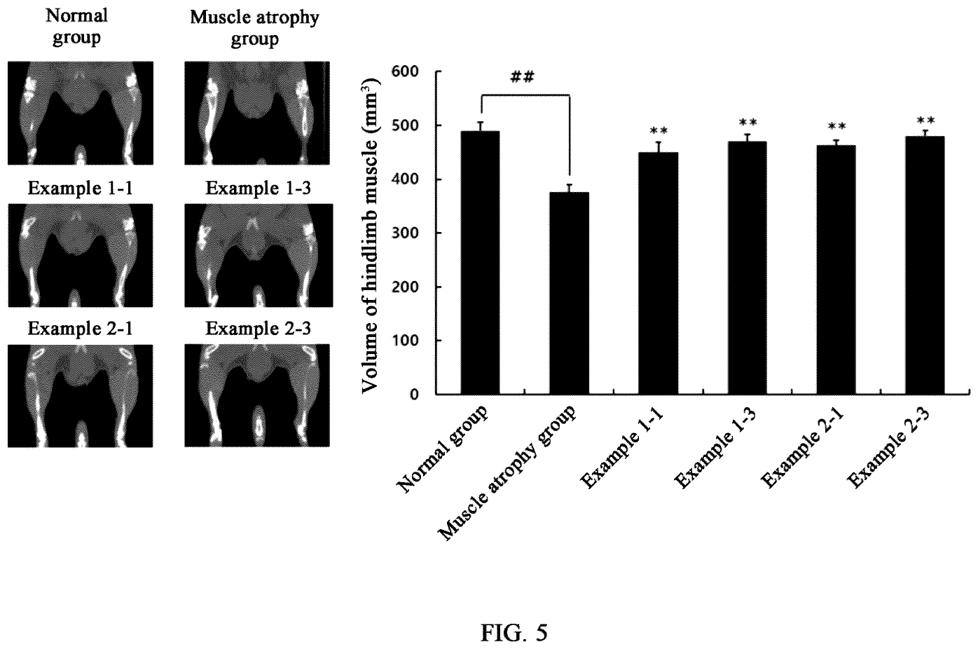

As a result of treating a mouse animal model of immobilization using a skin stapler with hot water and ethanol extracts of the aerial part of Korean mint or hot water and ethanol extracts of Korean mint leaf, muscular strength, muscle volume and muscle weight were increased significantly as compared to a muscle atrophy group (FIG. 5, Table 5 and Table 6). In addition, when the same animal model was treated with tilianin, muscular strength and muscle weight were increased significantly as compared to a muscle atrophy group.

The food composition for ameliorating and preventing muscular diseases or improving muscular functions of the present disclosure may be used to prevent or ameliorate muscular diseases caused by muscle wasting or degeneration. Muscle wasting and degeneration are caused by genetic factors, acquired factors, aging, etc., and muscle wasting is characterized by gradual loss of muscle mass and weakening and degeneration of muscles, particularly skeletal or voluntary muscles and cardiac muscles. Related diseases include sarcopenia, muscular atrophy, muscular dystrophy, atony, muscular degeneration, myasthenia, cachexia, etc. The composition of the present disclosure is effective in increasing muscle mass, regardless of the type of muscles.

The food composition of the present disclosure includes all types such as a functional food, a nutritional supplement, a functional health food, a food additive, a feed for human or animals including livestock. The food composition can be prepared into various forms according to common methods known in the art.

The food composition may be prepared into general foods, by adding the Korean mint extract to beverages (including alcoholic beverages), fruits and processed foods thereof (e.g., canned fruit, bottled fruit, jam, marmalade, etc.), fish, meat and processed foods thereof (e.g., ham, sausage, corned beef, etc.), bread and noodles (e.g., udon, buckwheat noodles, instant noodles, spaghetti, macaroni, etc.), fruit juice, various drinks, cookie, taffy, dairy products (e.g., butter, cheese, etc.), vegetable fats and oils, margarine, vegetable proteins, retort foods, frozen foods, various seasonings (e.g., soybean paste, soy sauce, sauce, etc.), etc., although not being limited thereto. In addition, the nutritional supplement may be prepared by adding the Korean mint extract to a capsule, a tablet, a pill, etc., although not being limited thereto. In addition, the functional health food may be prepared by preparing the Korean mint extract into a tea, a juice or a drink for drinking (health beverage) or into a liquid, a granule, a capsule or a powder for ingestion, although not being limited thereto. In addition, for use as the food additive, the Korean mint extract may be prepared into a powder or a concentrate. In addition, the Korean mint extract may be mixed with an active ingredient known to have the effect of ameliorating and improving muscular diseases or improving muscular functions for preparation into a composition.

When the composition for ameliorating and treating muscular diseases or improving muscular functions of the present disclosure is used as a health beverage composition, the health beverage composition may further contain various flavorants, natural carbohydrates, etc. as additional ingredients like common beverages. The natural carbohydrates may include monosaccharides such as glucose or fructose, disaccharides such as maltose or sucrose, polysaccharides such as dextrin or cyclodextrin and sugar alcohols such as xylitol, sorbitol, erythritol, etc. A natural sweetener such as thaumatin or stevia extract, a synthetic sweetener such as saccharin or aspartame, etc. may be used as a sweetener. In general, the natural carbohydrate is used in an amount of about 0.01-5.0 g, specifically about 0.1-1.0 g, per 100 mL of the composition of the present disclosure.

The Korean mint extract may be contained in the food composition for ameliorating or preventing muscular diseases or improving muscular functions as an active ingredient. It may be used in an amount effective for achieving the action of ameliorating and improving muscular diseases or improving muscular functions. Specifically, it may be used in an amount of 0.01-100 wt % based on the total weight of the composition, although not being specially limited thereto. The food composition of the present disclosure may be prepared by mixing the Korean mint extract with another active ingredient known to be effective for ameliorating and improving muscular diseases or improving muscular functions.

The composition for preventing and treating a muscular disease of the present disclosure may also be a pharmaceutical composition. When the composition for preventing and treating a muscular disease of the present disclosure is a pharmaceutical composition, it may be used to prevent or treat a muscular disease caused by muscle wasting or degeneration. Muscle wasting and degeneration are caused by genetic factors, acquired factors, aging, etc., and muscle wasting is characterized by gradual loss of muscle mass and weakening and degeneration of muscles, particularly skeletal or voluntary muscles and cardiac muscles. Related diseases include sarcopenia, muscular atrophy, muscular dystrophy, atony, muscular degeneration, myasthenia, cachexia, etc. The composition of the present disclosure is effective in increasing muscle mass, regardless of the type of muscles.

The pharmaceutical composition of the present disclosure may contain a pharmaceutically acceptable salt of tilianin. In the present specification, the term ‘pharmaceutically acceptable’ means that the salt is physiologically acceptable and usually does not cause allergic or similar reactions when administered to human. Specifically, the salt may be an acid addition salt formed from a pharmaceutically acceptable free acid.

The pharmaceutically acceptable salt of tilianin may be an acid addition salt formed from an organic acid or an inorganic acid. The organic acid includes, for example, formic acid, acetic acid, propionic acid, lactic acid, butyric acid, isobutyric acid, trifuloroacetic acid, malic acid, maleic acid, malonic acid, fumaric acid, succinic acid, succinic acid monoamide, glutamic acid, tartaric acid, oxalic acid, citric acid, glycolic acid, glucuronic acid, ascorbic acid, benzoic acid, phthalic acid, salicylic acid, anthranilic acid, dichloroacetic acid, aminooxyacetic acid, benzenesulfonic acid, p-toluenesulfonic acid or methanesulfonic acid. The inorganic acid includes, for example, hydrochloric acid, bromic acid, sulfuric acid, phosphoric acid, nitric acid, carbonic acid or boric acid. The acid addition salt may be specifically in the form of a hydrochloride or an acetate, more specifically in the form of a hydrochlorides.

The acid addition salt is prepared a general method of preparing a salt, i.e., a) by mixing tilianin directly with an acid, b) dissolving tilianin in an aqueous or nonaqueous solvent and then mixing with an acid, or c) adding tilianin to an acid in an aqueous or nonaqueous solvent and then mixing them.

In addition, the salt may also be a GABA salt, a gabapentin salt, a pregabalin salt, a nicotinate, an adipate, a hemimalonate, a cysteinate, an acetylcysteinate, a methionine salt, an arginate, a lysinate, an ornithine salt, an aspartate, etc.

In addition, the pharmaceutical composition for preventing and treating muscular diseases or improving muscular functions of the present disclosure may further contain a pharmaceutically acceptable carrier.

The pharmaceutically acceptable carrier may include, for example, a carrier for oral administration and a carrier for parenteral administration. The carrier for oral administration may include lactose, starch, a cellulose derivative, magnesium stearate, stearic acid, etc. And, the carrier for parenteral administration may include water, a suitable oil, saline, aqueous glucose, glycol, etc. In addition, a stabilizer and a preservative may be further included. Suitable stabilizers include an antioxidant such as sodium bisulfite, sodium sulfite or ascorbic acid. Suitable preservatives include benzalkonium chloride, methyl or propyl paraben and chlorobutanol. For other pharmaceutically acceptable carriers, those described in Remington's Pharmaceutical Sciences (19th ed., Mack Publishing Company, Easton, P A, 1995) may be referred to.

The pharmaceutical composition of the present disclosure may be administered to a mammal including human by any means. For example, it may be administered orally or parenterally. The parenteral administration may be made by intravenous, intramuscular, intraarterial, intramedullary, intradural, intracardiac, transdermal, subcutaneous, intraperitoneal, intranasal, intestinal, topical, sublingual or intrarectal administration, although not being limited thereto.

The pharmaceutical composition of the present disclosure may be prepared into a formulation for oral or parenteral administration depending on the administration route. The formulation may be prepared using one or more buffer (e.g., saline or PBS), antioxidant, bacteriostat, chelating agent (e.g., EDTA or glutathione), filler, extender, binder, adjuvant (e.g., aluminum hydroxide), suspending agent, thickener, wetting agent, disintegrant, surfactant, diluent or excipient.

Solid formulations for oral administration include a tablet, a pill, a powder, a granule, a liquid, a gel, a syrup, a slurry, a suspension, a capsule, etc. Such a solid formulation may be prepared by mixing the pharmaceutical composition of the present disclosure with at least one excipient, e.g., starch (including corn starch, wheat starch, rice starch, potato starch, etc.), calcium carbonate, sucrose, lactose, dextrose, sorbitol, mannitol, xylitol, erythritol, maltitol, cellulose, methyl cellulose, sodium carboxymethyl cellulose, hydroxypropyl methyl cellulose, gelatin, etc. For example, after mixing the active ingredient with a solid excipient, pulverizing the mixture and adding a suitable adjuvant, the mixture may be processed to a granule mixture to obtain a tablet or a sugar-coated tablet.

In addition to a simple excipient, a lubricant such as magnesium stearate and talc may also be used. Liquid formulations for oral administration include a suspension, a liquid for internal use, an emulsion, a syrup, etc. In addition to a commonly used simple diluent such as water or liquid paraffin, various excipients, e.g., a wetting agent, a sweetener, an aromatic, a preservative, etc. may be contained.

In addition, crosslinked polyvinylpyrrolidone, agar, alginic acid, sodium alginate, etc. may be added as a disintegrant and an anti-agglomerating agent, a lubricant, a wetting agent, a flavorant, an emulsifier, an antiseptic, etc. may be further contained.

For parenteral administration, the pharmaceutical composition of the present disclosure may be formulated into an injection, a transdermal formulation or an intranasal inhalant using a suitable pharmaceutical carrier according to methods known in the art. The injection should be sterilized and protected from contamination by microorganisms such as bacteria and fungi. A carrier suitable for an injection may be water, ethanol, a polyol (e.g., glycerol, propylene glycol, polyethylene glycol, etc.), a mixture thereof and/or a solvent or a dispersion medium including vegetable oil. More specifically, Hank's solution, Ringer's solution, triethanolamine-containing PBS (phosphate-buffered saline), sterile water for injection, or an isotonic solution such as 10% ethanol, 40% propylene glycol or 5% dextrose may be used as the carrier. In order to protect the injection from contamination by microorganisms, various antibacterial agents and antifungal agents such as paraben, chlorobutanol, phenol, sorbic acid, thimerosal, etc. may be further contained. In addition, the injection may further contain an isotonic agent such as a sugar or sodium chloride.

The formulations for transdermal administration includes an ointment, a cream, a lotion, a gel, a solution for external use, a paste, a liniment, an aerosol, etc. The ‘transdermal administration’ refers to delivery of an effective amount of the active ingredient contained in a pharmaceutical composition into skin by topically administering the pharmaceutical composition.

For administration by inhalation, the compound according to the present disclosure may be delivered conveniently in the form of an aerosol spray from a pressurized pack or a nebulizer using a suitable propellant, e.g., dichlorofluoromethane, trichlorofluoromethane, dichlorotetrafluoroethane, carbon dioxide or other suitable gas. For a pressurized aerosol, the dosage unit may be determined by providing a valve to deliver a metered amount. For example, the compound according to the present disclosure may be mixed with a suitable powder such as lactose or starch to form a gelatin capsule and a cartridge used in an inhaler or an insufflator. Formulations for parenteral administration are described in Remington's Pharmaceutical Science (15th edition, 1975. Mack Publishing Company, Easton, Pennsylvania 18042, Chapter 87: Blaug, Seymour).

The pharmaceutical composition for preventing and treating muscular diseases or improving muscular functions of the present disclosure can provide the desired effect of preventing and treating muscular diseases or improving muscular functions when it contains an effective amount of the Korean mint extract or tilianin. In the present specification, the ‘effective amount’ refers to an amount to induce a better response as compared to a negative control group, specifically an amount sufficient to improve muscular functions. The pharmaceutical composition of the present disclosure may contain 0.01-99.99% of the Korean mint extract or tilianin and a pharmaceutically acceptable carrier as the balance. The effective amount of the Korean mint extract or tilianin contained in the pharmaceutical composition of the present disclosure will vary depending on the form, etc. of the composition.

A total effective amount of the pharmaceutical composition of the present disclosure may be administered to a patient in a single dose, or may be administered to a patient in a multiple dose for a long period of time by a fractionated treatment protocol. The content of the active ingredient in the pharmaceutical composition of the present disclosure may vary depending on the severity of a disease. For parenteral administration, the Korean mint extract or tilianin may be administered once or several times a day, in an amount of 0.01-50 mg, more specifically 0.1-30 mg, per 1 kg of body weight. For oral administration, the Korean mint extract or tilianin may be administered once or several times a day, in an amount of 0.01-100 mg, more specifically 0.1-50 mg, per 1 kg of body weight. However, since the effective administration dosage of the Korean mint extract or tilianin is determined in consideration of various factors such as not only the administration route and number of the pharmaceutical composition but also the age, body weight, health condition and sex of a patient, the severity of a disease, diet, excretion rate, etc., those having ordinary knowledge in the art may determine the adequate effective administration dosage of the Korean mint extract or tilianin for the specific purpose of preventing and treating muscular diseases. The formulation, administration route and administration method of the pharmaceutical composition of the present disclosure are not particularly limited as long as the effect of the present disclosure is exerted.

The pharmaceutical composition for preventing and treating muscular diseases or improving muscular functions of the present disclosure may be used either alone or in combination with surgery, radiation therapy, hormone therapy, chemotherapy or a method using a biological response controller.

The pharmaceutical composition for preventing and treating muscular diseases or improving muscular functions of the present disclosure may also be provided as a formulation for external application, which contains the Korean mint extract or tilianin as an active ingredient.

When the pharmaceutical composition for preventing and treating muscular diseases or improving muscular functions of the present disclosure is used as a formulation for external application to skin, it may further contain an adjuvant commonly used in the field of dermatology such as a fatty substance, an organic solvent, a solubilizer, a concentrator, a gelling agent, a softening agent, an antioxidant, a suspending agent, a stabilizer, a foaming agent, an aromatic, a surfactant, water, an ionic emulsifier, a nonionic emulsifier, a filler, a metal ion sequestrant, a chelating agent, a preservative, a vitamin, a blocker, a wetting agent, an essential oil, a dye, a pigment, a hydrophilic activator, a lipophilic activator, a lipid vesicle, etc. These ingredients may be introduced in amounts that are generally used in the field of dermatology.

When the pharmaceutical composition for preventing and treating muscular diseases or improving muscular functions of the present disclosure is provided as a formulation for external application to skin, it may be in the form of an ointment, a patch, a gel, a cream, a spray, etc., although not being limited thereto.

The composition for ameliorating and treating muscular diseases or improving muscular functions of the present disclosure may also be a cosmetic composition. The cosmetic composition of the present disclosure contains the Korean mint extract or tilianin as an active ingredient and may be prepared into a basic cosmetic composition (toilet water, cream, essence, facial cleanser such as cleansing foam or cleansing water, pack or body oil), a color cosmetic composition (foundation, lipstick, mascara or makeup base), a hair product composition (shampoo, rinse, hair conditioner or hair gel), a soap, etc. together with a dermatologically acceptable excipient.

The excipient may include, for example, a skin softener, a skin permeation enhancer, a coloring agent, an aromatic, an emulsifier, a thickener and a solvent, although not being limited thereto. In addition, a flavorant, a colorant, a sterilizer, an antioxidant, an antiseptic, a moisturizer, etc. may be included further. For the purpose of improvement of physical properties, a thicker, a mineral, a synthetic polymer material, etc. may be included. For example, when the cosmetic composition of the present disclosure is prepared into a facial cleanser or a soap, it may be prepared easily by adding the Korean mint extract or tilianin to a common facial cleanser or soap base. When a cream is prepared, it may be prepared by adding the Korean mint extract or tilianin to a general oil-in-water (O/W) cream base. In addition, a flavorant, a chelating agent, a colorant, an antioxidant, an antiseptic, etc. and a synthetic or natural materials for improving physical properties, such as a protein, a mineral, a vitamin, etc. may be added.

The content of the Korean mint extract or tilianin in the cosmetic composition of the present disclosure may be specifically 0.001-10 wt %, more specifically 0.01-5 wt %, based on the total weight of the composition, although not being limited thereto. If the content is lower than 0.001 wt %, the desired effect cannot be expected. And, if it exceeds 10 wt %, there may occur problems in terms of safety or formulation stability.

The composition of the present disclosure may be added to a feed additive for preventing or ameliorating muscular diseases or a feed composition containing the same.

In the present disclosure, the term “feed additive” includes a substance added to a feed for various purposes such as nutritional supplement, prevention of body weight loss, enhancement of digestibility of cellulose in the feed, improvement of milk quality, prevention of reproductive failure and improvement of fertility, prevention of heat stress during the summer season, etc. The feed additive of the present disclosure corresponds to the supplementary feed defined in the Control of Livestock and Fish Feed Act, and may include minerals such as sodium bicarbonate, bentonite, magnesium oxide, complex minerals, etc., trace minerals such as zinc, copper, cobalt, selenium, etc., vitamins such as carotene, vitamin A, D and E, nicotinic acid, vitamin B complex, etc., amino acids such as methionine, lysine, etc., protected fatty acids such as fatty acid calcium, and probiotics such as lactic acid bacteria, yeast culture, fermented fungi, etc.

In the present disclosure, the term “feed” refers to any natural or artificial diet, meal, etc. or components of such meal intended to or suitable to be eaten, taken in and digested by animals. The feed including the composition for preventing or ameliorating muscular diseases according to the present disclosure as an active ingredient may be prepared into various types of feed known in the art, specifically a concentrated feed, a crude feed and/or a specialty feed, although not being limited thereto.

The concentrated feed includes seeds of grains such as wheat, oats, corn, etc., hulls of rice, wheat, barley, etc. as byproducts obtained after polishing of grains, seedcakes obtained as byproducts after extracting oil from bean, rape, sesame, linseed, coconut palm, etc., dregs remaining after extracting starch from sweet potato, potato, etc., animal feed such as fish meal, fish scraps, fish soluble obtained by concentrating a fresh liquid product obtained from fish, meat meal, blood meal, feather meal, skimmed milk powder, and dried whey obtained by drying whey which is a solution remaining when cheese and casein are produced from milk and skim milk, respectively, yeast, chlorella and marine algae, although not being limited thereto.

The crude feed includes fresh grass feed such as wild grass, pasture, fodder, etc., root vegetables such as turnip for feed, beet for feed, rutabaga which is a type of turnip, etc., silage which is a fodder obtained by fermenting fresh grass, fodder crops, grains, etc. filled in a silo with lactic acid bacteria, hay obtained by cutting and drying wild grass and pasture, straw of crops for breeding stock, and leaves of leguminous plants, although not being limited thereto. The specialty feed includes mineral feed such as oyster shells, rock salt, etc., urea feed such as urea or its derivatives such as isobutane diureide, etc., feed additives added in a trace amount to assorted feed so as to supplement lacking ingredients or to improve the storage property of the feed, and dietary supplements, although not being limited thereto.

The feed additive for preventing or ameliorating muscular diseases according to the present disclosure may be prepared by adding the Korean mint extract within an appropriate effective concentration range according to various methods for preparing feed known in the art.

The feed additive according to the present disclosure may be used for any subject requiring prevention or amelioration of muscular diseases. For example, it may be administered to any subject such as a non-human animal, e.g., cow, horse, pig, goat, sheep, dog, cat, rabbit, birds, fish, etc.

Hereinafter, the present disclosure is described in more detail through examples.

However, the following examples merely illustrate the present disclosure, and do not limit the present disclosure.

### [Example 1] Preparation of Extract of Aerial Part of Korean Mint

### <1-1> Preparation of Hot Water Extract of Aerial Part of Korean Mint

After pulverizing the dried aerial part of Korean mint with a mixer, 10 g of the aerial part of Korean mint sample was added to 100 ml of water and extracted for 3 hours at 80° C. The extracted sample was filtered with Whatman No. 1 filter paper under reduced pressure, and a hot water extract of the aerial part of Korean mint was obtained by removing the solvent by concentrating the filtered extract with a rotary vacuum evaporator.

### <1-2> Preparation of 50% Ethanol Extract of Aerial Part of Korean Mint

After pulverizing the dried aerial part of Korean mint with a mixer, 10 g of the aerial part of Korean mint sample was added to 100 mL of 50% ethanol and extracted for 3 hours at 40° C. The extracted sample was filtered with Whatman No. 1 filter paper under reduced pressure, and a 50% ethanol extract of the aerial part of Korean mint was obtained by removing the solvent by concentrating the filtered extract with a rotary vacuum evaporator.

### <1-3> Preparation of Aerial Part of 100% Ethanol Extract of Korean Mint

After pulverizing the dried aerial part of Korean mint with a mixer, 10 g of the aerial part of Korean mint sample was added to 100 ml of 100% ethanol and extracted for 3 hours at 40° C. The extracted sample was filtered with Whatman No. 1 filter paper under reduced pressure, and a 100% ethanol extract of the aerial part of Korean mint was obtained by removing the solvent by concentrating the filtered extract with a rotary vacuum evaporator.

### <1-4> Preparation of Ethyl Acetate Extract of Aerial Part of Korean Mint

After pulverizing the dried aerial part of Korean mint with a mixer, 10 g of the aerial part of Korean mint sample was added to 100 mL of ethyl acetate and extracted for 3 hours at 40° C. The extracted sample was filtered with Whatman No. 1 filter paper under reduced pressure, and an ethyl acetate extract of the aerial part of Korean mint was obtained by removing the solvent by concentrating the filtered extract with a rotary vacuum evaporator.

### <1-5> Preparation of Hexane Extract of Aerial Part of Korean Mint

After pulverizing the dried aerial part of Korean mint with a mixer, 10 g of the aerial part of Korean mint sample was added to 100 mL of hexane and extracted for 3 hours at 40° C. The extracted sample was filtered with Whatman No. 1 filter paper under reduced pressure, and a hexane extract of the aerial part of Korean mint was obtained by removing the solvent by concentrating the filtered extract with a rotary vacuum evaporator.

### <1-6> Preparation of Ultra-High-Pressure Extract of Aerial Part of Korean Mint

After pulverizing the dried aerial part of Korean mint with a mixer, 1 g of the pulverized aerial part of Korean mint and 76 mL of 18% ethanol were put in a polyethylene pack and extracted using an ultra-high-pressure extractor (Frescal MFP-7000; Mitsubishi Heavy Industries) after sealing. The ultra-high-pressure extraction condition was as follows: extraction pressure=320 MPa, extraction time=5 minutes. The extracted sample was filtered through Whatman No. 2 filter paper, and an ultra-high-pressure extract of the aerial part of Korean mint was obtained by removing the solvent by concentrating the filtered extract with a rotary vacuum evaporator.

### <1-7> Preparation of Subcritical Extract of Aerial Part of Korean Mint

After pulverizing the dried aerial part of Korean mint with a mixer, 50 g of the pulverized aerial part of Korean mint and 1 L of water were put in a subcritical water reactor of a subcritical extractor (Biovan, Gyeonggi, Korea). After sealing, the temperature of the reactor was raised to 200° C. When the temperature of the reactor reached 200° C., heating was stopped and extraction was conducted while maintaining the temperature for 20 minutes. 20 minutes later, the extract was transferred to a storage tank to which cooling water was supplied for rapid cooling to 30° C. Then, in order to separate the suspended residue, centrifugation was performed at 3,600 rpm for 30 minutes and only the supernatant was taken. A subcritical extract of the aerial part of Korean mint was obtained by completely removing the solvent using a freeze dryer (Ilshin Lab Co. Ltd., Seoul, Korea).

### <1-8> Preparation of Supercritical Extract of Aerial Part of Korean Mint

After pulverizing the dried aerial part of Korean mint with a mixer, 10 g of the pulverized aerial part of Korean mint was filled in a sample cartridge and extracted using a supercritical fluid extractor (SFX 3560, Isco Inc., Lincoln, NE, USA). The supercritical extraction condition was as follows: extraction pressure=300 bar, extraction temperature=50° C., flow rate of supercritical carbon dioxide=60 mL/min, extraction time=2 hours. After the supercritical fluid extraction was completed, a supercritical extract of the aerial part of Korean mint was obtained by lowering the pressure of the extractor.

### [Example 2] Preparation of Extract of Korean Mint Leaf

### <2-1> Preparation of Hot Water Extract of Korean Mint Leaf

After pulverizing dried Korean mint leaf with a mixer, 10 g of the Korean mint leaf sample was added to 100 ml of water and extracted for 3 hours at 80° C. The extracted sample was filtered with Whatman No. 1 filter paper under reduced pressure, and a hot water extract of Korean mint leaf was obtained by removing the solvent by concentrating the filtered extract with a rotary vacuum evaporator.

### <2-2> Preparation of 50% Ethanol Extract of Korean Mint Leaf

After pulverizing dried Korean mint leaf with a mixer, 10 g of the pulverized Korean mint leaf sample was added to 100 mL of 50% ethanol and extracted for 3 hours at 40° C. The extracted sample was filtered with Whatman No. 1 filter paper under reduced pressure, and a 50% ethanol extract of Korean mint leaf was obtained by removing the solvent by concentrating the filtered extract with a rotary vacuum evaporator.

### <2-3> Preparation of 100% Ethanol Extract of Korean Mint Leaf

After pulverizing dried Korean mint leaf with a mixer, 10 g of the pulverized Korean mint leaf sample was added to 100 mL of 100% ethanol and extracted for 3 hours at 40° C. The extracted sample was filtered with Whatman No. 1 filter paper under reduced pressure, and a 100% ethanol extract of Korean mint leaf was obtained by removing the solvent by concentrating the filtered extract with a rotary vacuum evaporator.

### <2-4> Preparation of Ethyl Acetate Extract of Korean Mint Leaf

After pulverizing dried Korean mint leaf with a mixer, 10 g of the pulverized Korean mint leaf sample was added to 100 mL of ethyl acetate and extracted for 3 hours at 40° C. The extracted sample was filtered with Whatman No. 1 filter paper under reduced pressure, and an ethyl acetate extract of Korean mint leaf was obtained by removing the solvent by concentrating the filtered extract with a rotary vacuum evaporator.

### <2-5> Preparation of Hexane Extract of Korean Mint Leaf

After pulverizing dried Korean mint leaf with a mixer, 10 g of the pulverized Korean mint leaf sample was added to 100 ml of hexane and extracted for 3 hours at 40° C. The extracted sample was filtered with Whatman No. 1 filter paper under reduced pressure, and a hexane extract of Korean mint leaf was obtained by removing the solvent by concentrating the filtered extract with a rotary vacuum evaporator.

### <2-6> Preparation of Ultra-High-Pressure Extract of Korean Mint Leaf

After pulverizing dried Korean mint leaf with a mixer, 1 g of the pulverized Korean mint leaf and 76 mL of 18% ethanol were put in a polyethylene pack and extracted using an ultra-high-pressure extractor (Frescal MFP-7000; Mitsubishi Heavy Industries) after sealing. The ultra-high-pressure extraction condition was as follows: extraction pressure=320 MPa, extraction time=5 minutes. The extracted sample was filtered through Whatman No. 2 filter paper, and an ultra-high-pressure extract of Korean mint leaf was obtained by removing the solvent by concentrating the filtered extract with a rotary vacuum evaporator.

### <2-7> Preparation of Subcritical Extract of Korean Mint Leaf

After pulverizing dried Korean mint leafs with a mixer, 50 g of the pulverized Korean mint leaf and 1 L of water were put in a subcritical water reactor of a subcritical extractor (Biovan, Gyeonggi, Korea). After sealing, the temperature of the reactor was raised to 200° C. When the temperature of the reactor reached 200° C., heating was stopped and extraction was conducted while maintaining the temperature for 20 minutes. 20 minutes later, the extract was transferred to a storage tank to which cooling water was supplied for rapid cooling to 30° C. Then, in order to separate the suspended residue, centrifugation was performed at 3,600 rpm for 30 minutes and only the supernatant was taken. A subcritical extract of Korean mint leaf was obtained by completely removing the solvent using a freeze dryer (Ilshin Lab Co. Ltd., Seoul, Korea).

### <2-8> Preparation of Supercritical Extract of Korean Mint Leaf

After pulverizing dried Korean mint leaf with a mixer, 10 g of the pulverized Korean mint leaf was filled in a sample cartridge and extracted using a supercritical fluid extractor (SFX 3560, Isco Inc., Lincoln, NE, USA). The supercritical extraction condition was as follows: extraction pressure=300 bar, extraction temperature=50° C., flow rate of supercritical carbon dioxide=60 mL/min, extraction time=2 hours. After the supercritical fluid extraction was completed, a supercritical extract of Korean mint leaf was obtained by lowering the pressure of the extractor.

### [Example 3] Effect of Increasing Activity of mTOR, Biomarker of Muscle Protein Synthesis, of Hot Water Extract of Aerial Part of Korean Mint

It is known that the activation of the mTOR protein by phosphorylation can induce muscle protein synthesis in muscle cells by the PI3K/Akt signaling pathway and can activate proteins involved in increase of muscle mass. Therefore, in order to investigate the activity of inducing muscle formation of Korean mint, the activity of mTOR was investigated using a mTOR sandwich ELISA kit (Cell Signaling Technology, Beverly, MA, USA).

L6 myoblasts (ATCC; Manassas, VA, USA) were seeded on a 6-well plate at 1×10⁵ cells/well with Dulbecco's modified Eagle's medium (DMEM; Hyclone) containing 10% fetal bovine serum (FBS; Hyclone, Logan, UT, USA) and then cultured for 24 hours. After the culturing, the medium was removed from the well and exchanged with DMEM

(Hyclone) containing 2% horse serum (HS; Hyclone). Then, the L6 cells were differentiated into myotubes by culturing further for 6 days. Subsequently, the cells were treated with the hot water extract of the aerial part of Korean mint of Example 1-1 was dissolved in DMEM (Hyclone) to 40 μg/mL or 80 μg/mL and cultured for 12 hours. After the culturing, the cells were lysed by treating with a cell lysis buffer. Proteins in the obtained cell lysate were quantified at a concentration of 1 mg/mL by Bradford assay (Bio-Rad Laboratories Inc., Hercules, CA, USA). 50 μL of the cell lysate was dispensed into a microwell attached with an anti-mTOR antibody and incubated at 37° C. for 2 hours. After washing 4 times with a washing buffer and treating with a detection antibody, the cell lysate was incubated at 37° C. for 1 hour. After washing again with a washing buffer for a total of 4 times and adding a horseradish peroxidase-conjugated secondary antibody, the cell lysate was incubated at 37° C. for 30 minutes. Finally, after washing 4 times with a washing buffer and adding a TMB substrate to each well, incubation was conducted at 37° C. for 10 minutes. Then, the TMB reaction was stopped by adding a stop solution. 2 minutes later, the mTOR level in the myotubes treated with the extract of Korean mint leaf was measured by measuring absorbance at 450 nm. The experiment was carried out in triplicate and the measurement result was represented as mean±standard deviation of percentage (%) with respect to a control group. The difference between groups was analyzed through Duncan's multiple range test by one-way analysis of variance using the SPSS25.0 statistical package (SPSS Inc., Chicago, IL, USA). A p value smaller than 5% was considered statistically significant.

As shown in FIG. 1, the treatment with the hot water extract of the aerial part of Korean mint resulted in significantly increased mTOR activity (**p<0.01) as compared to the control group not treated with the extract. This means that the hot water extract of the aerial part of Korean mint of the present disclosure has superior ability of promoting muscle protein synthesis in muscle cells.

### [Example 4] Effect of Increasing Activity of mTOR, Biomarker of Muscle Protein Synthesis, of Extracts of Aerial Part of Korean Mint

mTOR activity was measured in the same manner as in Example 3 except that the extracts of the aerial part of Korean mint of Example 1-2 to Example 1-8 were treated at 80 μg/mL.

As shown in Table 1, the treatment with the extracts of the aerial part of Korean mint resulted in significantly increased mTOR activity (**p<0.01) as compared to the control group not treated with the extract. This means that the extracts of the aerial part of Korean mint of the present disclosure have superior ability of promoting muscle protein synthesis in muscle cells.

TABLE 1

| Test groups | Relative mTOR activity (%) |
|---|---|
| Example 1-2 | 128.6 ± 3.0** |
| Example 1-3 | 132.9 ± 4.2** |
| Example 1-4 | 129.8 ± 3.8** |
| Example 1-5 | 133.1 ± 5.9** |
| Example 1-6 | 128.9 ± 3.8** |
| Example 1-7 | 131.7 ± 3.4** |
| Example 1-8 | 136.9 ± 4.7** |

### [Example 5] Effect of Inhibiting Expression of Muscle Protein Degradation Biomarkers of Hot Water Extract of Aerial Part of Korean Mint

L6 myoblasts (ATCC), which are muscle cells, were added to a 6-well plate with DMEM (Hyclone) containing 10% FBS (Hyclone) at 1×10⁵ cells/mL. When the cell density reached about 80-85%, the medium remaining in the well was removed and differentiation into myotubes was induced by treating the cells with DMEM (Hyclone) containing 2% HS (Hyclone). The differentiation was performed for a total of 6 days while replacing the medium with a fresh one every 2 days. After the differentiation, the cells were treated with the hot water extract of the aerial part of Korean mint prepared in Example 1-1 at 40 μg/mL or 80 μg/mL, which was dissolved in DMEM (Hyclone) containing 50 ng/ml tumor necrosis factor alpha (TNF-α; PeproTech, Rocky Hills, NJ, USA). 12 hours later, total RNA was isolated using a TRIzol reagent (Takara, Otsu, Japan). The isolated total RNA was quantified using NanoDrop 1000 (Thermo Fisher Scientific Inc., Waltham, MA, USA). cDNA was synthesized from 16 μL of the quantified RNAs using a reverse transcriptase premix (ELPIS-Biotech) and a PCR machine (Gene Amp PCR System 2700; Applied Biosystems, Foster City, CA, USA) under the condition of 42° C. for 55 minutes and 70° C. for 15 minutes. PCR was performed by repeating 30 cycles of 95° C. for 30 seconds, 60° C. for 1 minute and 72° C. for 1 minute using 1 μL of cDNA from 16 μL of the produced cDNA, specific primers described below (Bioneer, Daejeon, Korea) and a PCR premix (ELPIS-Biotech).

MuRF1
(SEQ ID NO 1)
Forward primer: 5′-CCGGACGGAAATGCTATGGA-3′
(SEQ ID NO 2)
Reverse primer: 5′-AGCCTGGAAGATGTCGTTGG-3′
Atrogin-1
(SEQ ID NO 3)
Forward primer: 5′-GTTACTGCAACAAGGAGAATCTGTT-3′
(SEQ ID NO 4)
Reverse primer: 5′-CCGTATGAGTCTTATGTTTTGCTGG-3′
β-Actin
(SEQ ID NO 5)
Forward primer: 5′-TGACAGGATGCAGAAGGAGATTAC-3′
(SEQ ID NO 6)
Reverse primer: 5′-TAAAACGCAGCTCAGTAACAGTC-3′

The cDNA amplified by the PCR was separated by electrophoresis on 1.5% agarose gel, and cDNA bands were identified using a G; BOX EF imaging system (Syngene).

As shown in FIG. 2, whereas the mRNA expression level of atrogin-1 and MuRF1 involved in muscle protein degradation was increased significantly (##p<0.01) by TNF-α, the treatment with the hot water extract of the aerial part of Korean mint significantly decreased the mRNA expression level of atrogin-1 and MuRF1 (*p<0.05, **p<0.01). This means that the hot water extract of the aerial part of Korean mint of the present disclosure has superior effect of inhibiting the expression of biomarkers involved in muscle protein degradation in muscle cells.

### [Example 6] Inhibition of Expression Level of Muscle Protein Degradation Biomarkers by Extracts of Aerial Part of Korean Mint

Experiment was carried out in triplicate in the same manner as in Example 5, except that the extracts of the aerial part of Korean mint of Example 1-2 to Example 1-8 were treated at 80 μg/mL. Then, after measuring the band density of MuRF1 and atrogin-1 mRNA using the ImageJ program (National Institute of Health, Bethesda, MD, USA), the measurement result was represented as mean±standard deviation of percentage (%) with respect to a control group.

As shown in Table 2, whereas the mRNA expression of atrogin-1 and MuRF1 was increased significantly (##p<0.01) by treatment with TNF-α, the treatment with the extracts of the aerial part of Korean mint significantly decreased the mRNA expression level as compared to the TNF-α treatment group (**p<0.01). This means that the extracts of the aerial part of Korean mint of the present disclosure have superior effect of inhibiting muscle protein degradation in muscle cells.

TABLE 2

| | Relative MuRF1 | Relative atrogin-1 |
|---|---|---|
| Test groups | expression level (%) | expression level (%) |
| TNF-α | 268.9 ± 29.5^{##} | 423.4 ± 48.4^{##} |
| Example 1-2 | 128.4 ± 37.8** | 163.6 ± 40.9** |
| Example 1-3 | 119.3 ± 35.1** | 156.9 ± 36.9** |
| Example 1-4 | 112.5 ± 40.9** | 167.0 ± 33.8** |
| Example 1-5 | 109.4 ± 27.4** | 170.2 ± 43.2** |
| Example 1-6 | 119.8 ± 36.0** | 159.4 ± 38.9** |
| Example 1-7 | 110.2 ± 37.2** | 163.6 ± 28.6** |
| Example 1-8 | 104.7 ± 30.0** | 148.0 ± 33.4** |

### [Example 7] Increased Activity of Muscle Protein Synthesis Biomarker mTOR by Extracts of Korean Mint Leaf

mTOR activity was measured in the same manner as in Example 1, except that each of the extracts of Korean mint leaf of Example 2-1 to Example 2-8 was treated at 80 μg/mL.

As shown in Table 3, the treatment with the extracts of Korean mint leaf resulted in significantly increased mTOR activity (**p<0.01) as compared to the control group not treated with the extract. This means that the extracts of Korean mint leaf of the present disclosure have superior ability of promoting muscle protein synthesis in muscle cells.

TABLE 3

| Test groups | Relative mTOR activity (%) |
|---|---|
| Example 2-1 | 138.8 ± 7.4** |
| Example 2-2 | 140.3 ± 5.9** |
| Example 2-3 | 146.7 ± 4.3** |
| Example 2-4 | 142.6 ± 4.6** |
| Example 2-5 | 147.9 ± 6.0** |
| Example 2-6 | 138.4 ± 5.2** |
| Example 2-7 | 143.2 ± 8.4** |
| Example 2-8 | 147.9 ± 6.3** |

### [Example 8] Inhibition of Expression of Muscle Protein Degradation Biomarkers by Hot Water Extract of Aerial Part of Korean Mint

Experiment was carried out in triplicate in the same manner as in Example 5, except that the extracts of Korean mint leaf of Example 2-1 to Example 2-8 were treated at 80 μg/mL. Then, after measuring the band density of MuRF1 and atrogin-1 mRNA using the ImageJ program (National Institute of Health, Bethesda, MD, USA), the measurement result was represented as mean±standard deviation of percentage (%) with respect to a control group.

As shown in Table 4, whereas the mRNA expression of atrogin-1 and MuRF1 was increased significantly (##p<0.01) by treatment with TNF-α, the treatment with the extracts of Korean mint leaf significantly decreased the mRNA expression level as compared to the TNF-α treatment group (**p<0.01). This means that the extracts of Korean mint leaf of the present disclosure have superior effect of inhibiting muscle protein degradation in muscle cells.

TABLE 4

| | Relative MuRF1 | Relative atrogin-1 |
|---|---|---|
| Test groups | expression level (%) | expression level (%) |
| TNF-α | 319.3 ± 40.2^{##} | 416.9 ± 45.4^{##} |
| Example 2-1 | 167.2 ± 36.9** | 212.5 ± 40.1** |
| Example 2-2 | 160.4 ± 42.7** | 200.4 ± 33.2** |
| Example 2-3 | 149.0 ± 47.1** | 185.2 ± 39.4** |
| Example 2-4 | 154.1 ± 27.0** | 209.8 ± 40.2** |
| Example 2-5 | 150.0 ± 45.9** | 198.0 ± 33.9** |
| Example 2-6 | 159.1 ± 40.2** | 179.3 ± 28.3** |
| Example 2-7 | 161.5 ± 35.3** | 183.1 ± 32.6** |
| Example 2-8 | 150.6 ± 46.4** | 170.4 ± 40.5** |

### [Example 9] Increased Expression of Muscle Protein Synthesis Biomarker by Tilianin

Experiment was carried out in triplicate in the same manner as in Example 3, except that the cells were treated with 20 μM or 40 μM tilianin.

As shown in FIG. 3, the treatment with tilianin resulted in significantly increased mTOR activity (**p<0.01) as compared to the control group. This means that the tilianin of the present disclosure has superior effect of promoting muscle protein synthesis in muscle cells.

### [Example 10] Inhibited Expression of Muscle Protein Degradation Biomarkers by Tilianin

Experiment was carried out in triplicate in the same manner as in Example 5, except that the cells were treated with 20 UM or 40 UM tilianin.

As shown in FIG. 4, the treatment with tilianin resulted in decreased mRNA of atrogin-1 and MuRF1, which was increased by TNF-α treatment, as compared to the control group. This means that the tilianin of the present disclosure has superior effect of inhibiting muscle protein degradation in muscle cells.

### [Example 11] Improvement of Muscular Functions and Muscle Mass in Animal Model by Korean Mint Extract

### <11-1> Animal Breeding and Induction of Muscle Atrophy

7-week-old male mice (C57BL/6J; DBL, Korea) were purchased as experimental animals. The breeding environment was maintained at a temperature of 23±2° C. and a relative humidity of 55±10%. Before the start of the experiment, a total of 20 mice were randomly divided into groups of 5 mice each. After a week of acclimation, anesthesia was induced by intraperitoneal injection of 325 mg/kg tribromoethanol (Sigma-Aldrich). After the anesthesia, the right hindlimb gastrocnemius muscle and the right sole of the mice in a muscle atrophy group and a sample administration group were stapled using a skin stapler (Unidus, Chungcheongbuk-do, Korea) to damage the muscle and keep the right hindlimb from moving. This state was maintained for a week. After one week, the stapes fixed to the gastrocnemius muscle and the sole were removed and the hot water extract of the aerial part of Korean mint of Example 1-1 (200 mg/kg), the ethanol extract of the aerial part of Korean mint of Example 1-3 (200 mg/kg), the hot water extract of Korean mint leaf of Example 2-1 (200 mg/kg) or the ethanol extract of Korean mint leaf of Example 2-3 (200 mg/kg) was orally administered every day for a week. For a normal group and the muscle atrophy group, saline was orally administered instead of the sample.

### <11-2> Improvement of Muscular Strength by Korean Mint Extract

After the end of the oral administration period, the muscular strength of the mice was measured using a muscular strength meter (Panlab, Barcelona, Spain). The tail of the mouse was pulled with constant force until the mouse released the bar of the muscular strength meter. A total of 5 consecutive tests were carried out per mouse.

As shown in Table 5, whereas the muscular strength was decreased significantly (##p<0.01) in the muscle atrophy group as compared to the normal group, the treatment with the Korean mint extract at 200 mg/kg resulted in significant increase of muscular strength as compared to the muscle atrophy group (**p<0.01). This means that the Korean mint extract of the present disclosure has superior effect of increasing muscular strength that has been decreased due to muscle atrophy.

TABLE 5

| Test groups | Muscular strength (g) |
|---|---|
| Normal group | 269.3 ± 15.2 |
| Muscle atrophy group | 181.6 ± 10.2^{##} |
| Example 1-1 | 231.2 ± 8.4** |
| Example 1-3 | 242.0 ± 11.4** |
| Example 2-1 | 236.1 ± 14.2** |
| Example 2-3 | 244.7 ± 9.9** |

### <11-3> Increase of Muscle Volume by Korean Mint Extract

After anesthetizing the mice with isoflurane, the volume and density of the right hindlimb muscle were measured by positron emission tomography/computed tomography/single photon emission tomography (microPET/CT/SPECT; Siemens Inveon, Knoxville, TN, USA).

As shown in FIG. 6, whereas the muscle volume was decreased significantly (##p<0.01) in the muscle atrophy group as compared to the normal group, the treatment with the Korean mint extract at 200 mg/kg resulted in significant increase of muscle volume as compared to the muscle atrophy group (**p<0.01). This means that the Korean mint extract of the present disclosure has superior effect of increasing muscle volume that has been decreased due to muscle atrophy.

### <11-4> Increase of Muscle Weight by Korean Mint Extract

After the muscular strength measurement was finished, the experimental animal was anesthetized by intraperitoneal injection of 325 mg/kg tribromoethanol (Sigma-Aldrich) and then sacrificed by cardiac puncture. After confirming that heartbeat was stopped, undamaged tibialis anterior muscle was extracted from the right hindlimb and then weighed.

As shown in Table 6, whereas the weight of the tibialis anterior muscle was decreased significantly in the muscle atrophy group as compared to the normal group, the treatment with the Korean mint extract at 200 mg/kg resulted in significant increase of the weight of the tibialis anterior muscle as compared to the muscle atrophy group (**p<0.01). This means that the Korean mint extract of the present disclosure has superior effect of increasing muscle weight that has been decreased due to muscle atrophy.

TABLE 6

| Test groups | Muscle weight (mg) |
|---|---|
| Normal group | 56.8 ± 2.8 |
| Muscle atrophy group | 46.2 ± 2.7^{##} |
| Example 1-1 | 53.7 ± 3.0** |
| Example 1-3 | 55.3 ± 3.3** |
| Example 2-1 | 54.4 ± 2.7** |
| Example 2-3 | 55.0 ± 3.5** |

### [Example 13] Improvement of Muscular Functions and Muscle Mass in Animal Model by Tilianin

After inducing muscle atrophy in the same manner as in Example 12-1, tilianin was orally administered every day at a concentration of 20 mg/kg. Then, muscular strength and muscle weight were measured in the same manner as in Example 12-2 and Example 12-3. As a result, muscular strength and muscle weight were increased in the muscle atrophy group as compared to the tilianin administration group by 30.3%(**p<0.01) and 15.2%(**p<0.01), respectively. This means that the tilianin of the present disclosure has superior effect of increasing muscular strength and muscle weight that have been decreased due to muscle atrophy.

Hereinafter, preparation examples of pharmaceutical drugs, foods or cosmetics containing the Korean mint extract or tilianin according to the present disclosure as an active ingredient are described. However, they are not intended to limit the scope of the present disclosure. Pharmaceutical drug, food or cosmetic compositions of Preparation Examples 1-3 were prepared according to common methods using the Korean mint extract or tilianin having superior effect of ameliorating, treating or preventing muscular diseases or improving muscular functions.

### [Preparation Example 1] Pharmaceutical Drugs

### <1-1> Powder

After mixing 50 mg of the Korean mint extract or tilianin of the present disclosure and 2 g of crystalline cellulose, a powder was prepared by filling the mixture in an airtight pouch.

### <1-2> Tablet

After mixing 50 mg of the Korean mint extract or tilianin of the present disclosure, 400 mg of crystalline cellulose and 5 mg of magnesium stearate, a tablet was prepared according to a common tableting method.

### <1-3> Capsule

After mixing 30 mg of the Korean mint extract or tilianin of the present disclosure, 100 mg of whey protein, 400 mg of crystalline cellulose and 6 mg of magnesium stearate, a capsule was prepared by filling the mixture in a gelatin capsule according to a common method.

### [Preparation Example 2] Foods

### <2-1> Health Food

1000 mg of the Korean mint extract of the present disclosure was mixed with 70 μg of vitamin A acetate, 1.0 mg of vitamin E, 0.13 mg of vitamin B1, 0.15 mg of vitamin B2, 0.5 mg of vitamin B6, 0.2 μg of vitamin B12, vitamin C 10 mg, 10 μg of biotin, 1.7 mg of nicotinamide, 50 μg of folic acid, 0.5 mg of calcium pantothenate, 1.75 mg of ferrous sulfate, 0.82 mg of zinc oxide, 25.3 mg of magnesium carbonate, 15 mg of monopotassium phosphate, 55 mg of dicalcium phosphate, 90 mg of potassium citrate, 100 mg of calcium carbonate and 24.8 mg of magnesium chloride. After mixing the above ingredients and preparing a granule therefrom, a health food composition was prepared according to a common method.

### <2-2> Health Beverage

After mixing 1000 mg of the Korean mint extract of the present disclosure, 1000 mg of citric acid, 100 g of oligosaccharide, 2 g of plum concentrate and 1 g of taurine and adding purified water to make a total volume of 900 mL, followed by heating for about 1 hour at 85° C. under stirring, the prepared solution was filtered, taken in a sterilized 2-L container, sealed, sterilized and then stored in a refrigerator until use for preparation of a health beverage composition.

### <2-3> Chewing Gum

A chewing gum was prepared according to a common method by mixing 20 wt % of gum base, 76.9 wt % of sugar, 1 wt % of a flavorant and 2 wt % of water with 0.1 wt % of the Korean mint extract of the present disclosure.

### <2-4> Candy

A candy was prepared according to a common method by mixing 60 wt % of sugar, 39.8 wt % of starch syrup and 0.1 wt % of a flavorant with 0.1 wt % of the Korean mint extract of the present disclosure.

### <2-5> Biscuit

A biscuit was prepared according to a common method by mixing 25.59 wt % of soft wheat flour, 22.22 wt % of plain wheat flour, 4.80 wt % of refined sugar, 0.73 wt % of table salt, 0.78 wt % of glucose, 11.78 wt % of palm shortening, 1.54 wt % of ammonium, 0.17 wt % of sodium bicarbonate, 0.16 wt % of sodium bisulfite, 1.45 wt % of rice flour, 0.0001 wt % of vitamin B, 0.04 wt % of milk flavor, 20.6998 wt % of water, 1.16 wt % of whole milk powder, 0.29 wt % of substitute milk powder, 0.03 wt % of monocalcium phosphate, 0.29 wt % of spray salt and 7.27 wt % of oil mist with 0.8301 wt % of the Korean mint extract of the present disclosure.

### [Preparation Example 3] Cosmetics

### <3-1> Nourishing Lotion (Milk Lotion)

A nourishing lotion was prepared using the Korean mint extract of the present disclosure by a common method according to the composition described in Table 7.

TABLE 7

| Ingredients | Preparation Example 3-1 (wt %) |
|---|---|
| Korean mint extract | 2.0 |
| Squalane | 5.0 |
| Beeswax | 4.0 |
| Polysorbate 60 | 1.5 |
| Sorbitan sesquioleate | 1.5 |
| Liquid paraffin | 0.5 |
| Caprylic/capric triglyceride | 5.0 |
| Glycerin | 3.0 |
| Butylene glycol | 3.0 |
| Propylene glycol | 3.0 |
| Carboxyvinyl polymer | 0.1 |
| Triethanolamine | 0.2 |
| Antiseptic, colorant and flavorant | Adequate |
| Purified water | To 100 |

### <3-2> Softening Lotion (Skin Lotion)

A softening lotion was prepared using the Korean mint extract of the present disclosure by a common method according to the composition described in Table 8.

TABLE 8

| Ingredients | Preparation Example 3-2 (wt %) |
|---|---|
| Korean mint extract | 2.0 |
| Glycerin | 3.0 |
| Butylene glycol | 2.0 |
| Propylene glycol | 2.0 |
| Carboxyvinyl polymer | 0.1 |
| PEG 12 nonyl phenyl ether | 0.2 |
| Polysorbate 80 | 0.4 |
| Ethanol | 10.0 |
| Triethanolamine | 0.1 |
| Antiseptic, colorant and flavorant | Adequate |
| Purified water | To 100 |

### <3-3> Nourishing Cream

A nourishing cream was prepared using the Korean mint extract of the present disclosure by a common method according to the composition described in Table 9.

TABLE 9

| | Preparation |
|---|---|
| Ingredients | Example 3-3 (wt %) |
| Korean mint extract | 2.0 |
| Polysorbate 60 | 1.5 |
| Sorbitan sesquioleate | 0.5 |
| PEG 60 hydrogenated castor oil | 2.0 |
| Liquid paraffin | 10 |
| Squalane | 5.0 |
| Caprylic/capric triglyceride | 5.0 |
| Glycerin | 5.0 |
| Butylene glycol | 3.0 |
| Propylene glycol | 3.0 |
| Triethanolamine | 0.2 |
| Antiseptic | Adequate |
| Colorant | Adequate |
| Flavorant | Adequate |
| Purified water | To 100 |

### <3-4> Massage Cream

A massage cream was prepared using the Korean mint extract of the present disclosure by a common method according to the composition described in Table 10.

TABLE 10

| Ingredients | Preparation Example 3-4 (wt %) |
|---|---|
| Korean mint extract | 1.0 |
| Beeswax | 10.0 |
| Polysorbate 60 | 1.5 |
| PEG 60 hydrogenated castor oil | 2.0 |
| Sorbitan sesquioleate | 0.8 |
| Liquid paraffin | 40.0 |
| Squalane | 5.0 |
| Caprylic/capric triglyceride | 4.0 |
| Glycerin | 5.0 |
| Butylene glycol | 3.0 |
| propylene glycol | 3.0 |
| Triethanolamine | 0.2 |
| Antiseptic, colorant and flavorant | Adequate |
| Purified water | To 100 |

### <3-5> Pack

A pack was prepared using the Korean mint extract of the present disclosure by a common method according to the composition described in Table 11.

TABLE 11

| Ingredients | Preparation Example 3-5 (wt %) |
|---|---|
| Korean mint extract | 1.0 |
| Polyvinyl alcohol | 13.0 |
| Sodium carboxymethyl cellulose | 0.2 |
| Glycerin | 5.0 |
| Allantoin | 0.1 |
| Ethanol | 6.0 |
| PEG 12 nonyl phenyl ether | 0.3 |
| Polysorbate 60 | 0.3 |
| Antiseptic, colorant and flavorant | Adequate |
| Purified water | To 100 |

### <3-6> Gel

A gel was prepared using the Korean mint extract of the present disclosure by a common method according to the composition described in Table 12.

TABLE 12

| Ingredients | Preparation Example 3-6 (wt %) |
|---|---|
| Korean mint extract | 0.5 |
| Sodium ethylenediaminetetraacetate | 0.05 |
| Glycerin | 5.0 |
| Carboxyvinyl polymer | 0.3 |
| Ethanol | 5.0 |
| PEG 60 hydrogenated castor oil | 0.5 |
| Triethanolamine | 0.3 |
| Antiseptic, colorant and flavorant | Adequate |
| Purified water | To 100 |

In the above tables, ‘Adequate’ means that an adequate amount may be added depending on preference, and ‘To 100’ means that the amount of purified water may is determined by subtracting the contents of other ingredients from 100 wt %.

### INDUSTRIAL APPLICABILITY

As described above, the present disclosure provides a composition for alleviating, treating or preventing muscular diseases or improving muscular functions, which contains a Korean mint extract or tilianin. More particularly, the Korean mint extract or tilianin of the present disclosure exhibits superior effect of ameliorating, treating or preventing muscular diseases or improving muscular functions by improving muscle mass and muscular strength through increased mTOR activity and decreased mRNA expression level of MuRF1 and atrogin-1. Accordingly, the present disclosure is highly applicable industrially because it can provide a composition exhibiting excellent effect of ameliorating, treating or preventing muscular diseases or improving muscular functions.

We claim:

1. A method for treating or preventing a muscular disease in a subject in need thereof, comprising:
administering a composition to the subject,
wherein the composition comprises a Korean mint extract or tilianin represented by Chemical Formula 1 as an active ingredient:

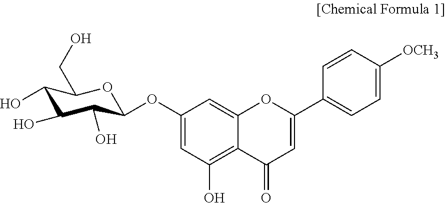

2. The method according to claim 1, wherein the Korean mint extract is obtained by extracting Korean mint with one or more solvent selected from a group consisting of water, a C₁₋₆ organic solvent, an ultra-high-pressure subcritical fluid and a supercritical fluid.

3. The method according to claim 2, wherein the organic solvent is at least one solvent selected from a group consisting of a C₁₋₆ alcohol, acetone, ether, benzene, chloroform, ethyl acetate, methylene chloride, hexane, cyclohexane and petroleum ether.

4. The method according to claim 1, wherein the muscular disease is one or more disease selected from a group consisting of sarcopenia, muscular atrophy, muscular dystrophy, atony, muscular degeneration, myasthenia and cachexia.

5. The method according to claim 1, wherein the composition is a food composition, a pharmaceutical composition, a cosmetic composition, or a feed composition.

6. A method for improving muscle mass and muscular strength in a subject in need thereof, comprising:
administering a composition to the subject,
wherein the composition comprising a Korean mint extract or tilianin represented by Chemical Formula 1 as an active ingredient:

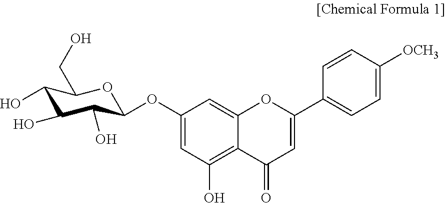

7. (canceled)
